(19) United States
(12) Patent Application Publication
(10) Pub. No.: US 2007/0089518 A1
(43) Pub. Date: Apr. 26, 2007

(54) Method and apparatus for orthopedic implant assessment

(76) Inventors: Milton Nance Ericson, Knoxville, TN (US); Charles Lanier Britton, Alcoa, TN (US); Justin Shekwoga Baba, Knoxville, TN (US); Robert J. Warmack, Knoxville, TN (US)

Correspondence Address: JOHN BRUCKNER PC, P.O. BOX 490, FLAGSTAFF, AZ, 86002-0490, US

(21) Appl. No.: 11/259,815

(22) Filed: Oct. 26, 2005

(51) Int. Cl. A61F 2/30; G01M 19/00; G01H 11/00

(52) U.S. Cl. 73/649; 73/172; 623/914

(57) ABSTRACT

Methods and apparatus are described for orthopedic implant assessment. A method includes characterizing wear of an orthopedic implant including measuring a dimension in a direction that defines a path that passes through an articulating surface of a wear element of the orthopedic implant using at least one thickness sensor. An apparatus includes an orthopedic implant including a wear element having an articulating surface; and at least one thickness sensor coupled to the wear element, the at least one thickness sensor measuring a dimension in a direction that defines a path that passes through the articulating surface of the wear element. A method includes characterizing forces within an orthopedic implant including using a plurality of individually addressable pressure sensors including measuring parasitic impedance between at least two of the plurality of individually addressable pressure sensors.

### STATEMENT AS TO RIGHTS TO INVENTIONS MADE UNDER FEDERALLY-SPONSORED RESEARCH OR DEVELOPMENT

This invention was made with United States Government support under prime contract No. DE-AC05-00OR22725 to UT-Battelle, L.L.C. awarded by the Department of Energy. The Government has certain rights in this invention.

### BACKGROUND INFORMATION

1. Field of the Invention

Embodiments of the invention relate generally to the field of orthopedic implants. More particularly, an embodiment of the invention relates to methods and apparatus for orthopedic implant assessment.

2. Discussion of the Related Art

Advances in surgical techniques and materials have enabled widespread use of complete joint replacements for knees and hips. Though improving, all friction surfaces in orthopedic implants experience load dependent wear that ultimately limits the useful lifetime of the device. Replacement of a worn artificial joint, though possible, is generally avoided, resulting in more limited application of these therapies. For example, joint replacements are often delayed so that the life expectancy of the recipient and the artificial joint are approximately correlated.

Significant research is underway in many commercial and research laboratories to improve the useable lifetime of orthopedic implants through better materials design, simulation models, and advanced techniques for modular replacement of worn friction surfaces. An enabling part of this research is the ability to monitor the implant in terms of load and wear. To date, reported methods for implant condition assessment include external radiometric and vibration-based techniques, or implanted orthopedic devices incorporating strain gauge techniques for force monitoring [1,2]. In addition, an implantable technique employing MEMs-based sensors for detection and elimination of bacterial bio-films has also been reported [3]. However, these reported implanted techniques do not enable direct wear measurement, and use very few sensors allowing only an integrated (i.e., not highly pixelated) assessment of force in the joint. What is needed is an alternative technique enabling accurate measurement of direct wear and force parameters that can be incorporated into both research and clinical implants for continuous or periodic wear and load assessment.

Heretofore, the requirements of joint wear measurement, and highly pixilated assessment of forces in the joint referred to above have not been fully met. What is needed is a solution that solves these problems.

### SUMMARY OF THE INVENTION

There is a need for the following embodiments of the invention. Of course, the invention is not limited to these embodiments.

According to an embodiment of the invention, a method comprises: characterizing wear of an orthopedic implant including measuring a dimension in a direction that defines a path that passes through an articulating surface of a wear element of the orthopedic implant using at least one thickness sensor. According to another embodiment of the invention, an apparatus comprises: an orthopedic implant including a wear element having an articulating surface; and at least one thickness sensor coupled to the wear element, the at least one thickness sensor measuring a dimension in a direction that defines a path that passes through the articulating surface of the wear element. According to another embodiment of the invention, a method comprises characterizing forces within an orthopedic implant including using a plurality of individually addressable pressure sensors including measuring parasitic impedance between at least two of the plurality of individually addressable pressure sensors.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of an embodiment of the invention without departing from the spirit thereof, and embodiments of the invention include all such substitutions, modifications, additions and/or rearrangements.

### BRIEF DESCRIPTION OF THE DRAWINGS

The drawings accompanying and forming part of this specification are included to depict certain embodiments of the invention. A clearer conception of embodiments of the invention, and of the components combinable with, and operation of systems provided with, embodiments of the invention, will become more readily apparent by referring to the exemplary, and therefore nonlimiting, embodiments illustrated in the drawings, wherein identical reference numerals (if they occur in more than one view) designate the same elements. Embodiments of the invention may be better understood by reference to one or more of these drawings in combination with the description presented herein. It should be noted that the features illustrated in the drawings are not necessarily drawn to scale.

FIG. 1 is a view of a capacitance sensor array enabling pixelated polymer thickness monitoring measurement, representing an embodiment of the invention.

FIG. 2 is an elevational view of a sensor system placement in tibial plate, representing an embodiment of the invention.

FIG. 3 is an isometric view of a configuration for capacitance-based measurement of wear and force monitoring, representing an embodiment of the invention.

FIG. 4A is a circuit schematic diagram of a non-inverting voltage readout amplifier configuration, representing an embodiment of the invention.

FIG. 4B is a circuit schematic diagram of an inverting voltage readout amplifier configuration, representing an embodiment of the invention.

FIG. 4C is a circuit schematic diagram of a floating readout amplifier configuration, representing an embodiment of the invention.

FIG. 5A is a block schematic diagram of an optical thickness sensor configuration, representing an embodiment of the invention.

FIG. 5B is a block schematic diagram of another optical thickness sensor configuration, representing an embodiment of the invention.

FIG. 6A is a circuit schematic diagram of a pixelated plate driven configuration, representing an embodiment of the invention.

FIG. 6B is a circuit schematic diagram of a common plate driven configuration, representing an embodiment of the invention.

### DESCRIPTION OF PREFERRED EMBODIMENTS

Embodiments of the invention and the various features and advantageous details thereof are explained more fully with reference to the nonlimiting embodiments that are illustrated in the accompanying drawings and detailed in the following description. Descriptions of well known starting materials, processing techniques, components and equipment are omitted so as not to unnecessarily obscure the embodiments of the invention in detail. It should be understood, however, that the detailed description and the specific examples, while indicating preferred embodiments of the invention, are given by way of illustration only and not by way of limitation. Various substitutions, modifications, additions and/or rearrangements within the spirit and/or scope of the underlying inventive concept will become apparent to those skilled in the art from this disclosure.

Within this application several publications are referenced by Arabic numerals, or principal author's name followed by year of publication, within parentheses or brackets. Full citations for these, and other, publications may be found at the end of the specification immediately preceding the claims after the section heading References. The disclosures of all these publications in their entireties are hereby expressly incorporated by reference herein for the purpose of indicating the background of embodiments of the invention and illustrating the state of the art.

The below-referenced U.S. Patent discloses embodiments that are useful for the purposes for which they are intended. The entire contents of U.S. Pat. No. 5,197,488 are hereby expressly incorporated by reference herein for all purposes.

The invention is a technique enabling accurate measurement of direct wear and force parameters that can be incorporated into both research and clinical implants for continuous or periodic wear and load assessment. In addition, the invention can incorporate different sensor types allowing monitoring of surrounding physiological parameters including tissue encapsulation, bone condition, osteointegration status including implant loosening, and the presence of infection. The invention is suitable for use with many different implant types including artificial knee (tibial plate, patella), hip, shoulder, and elbow joints, and may find use in spinal or other applications where bone is involved.

The invention can include high-resolution monitoring of both forces in prosthetic devices and the associated polymer-metal surface wear (one of the primary long-term failure mechanism in orthopedic implant devices). The invention can include direct wear measurement, including loss of material in the joint or deformation resulting in thin spots. The invention can be highly miniaturized and biocompatible, making it suitable for complete integration into existing prosthetic devices. The invention can include using low-power integrated circuits for sensing and telemetry allowing the sensor to be configured in a number of different ways, thereby enabling real-time continuous reporting, periodic reporting, and/or reporting only when requested. The invention can include different sensor types including capacitance-based, piezo-based, inductive, ultrasonic, MEMs-based pressure sensors, temperature sensors, vibration sensors and optical sensors will allow complete monitoring of wear, pressure, temperature, and surrounding physiological parameters including tissue encapsulation, bone condition, osteointegration status including implant loosening and the presence of infection.

The invention can include a capacitance-based sensing technique yielding direct wear measurement including capacitive dielectric enhancement and a dual-use sensor plate concept. The invention can include time-domain-reflectrometry and acoustic measurement techniques. The invention can include acoustic telemetry. The invention can include photonics-based sensing for infection, scarring, and bone condition monitoring. The invention can include MEMs-based sensors.

Sensing Method

This section describes a preferred sensing method and provides an overview of a preferred implant system including data telemetry. The sensing approach can involve placement of a capacitive sensor array in the polymer portion of a tibial plate.

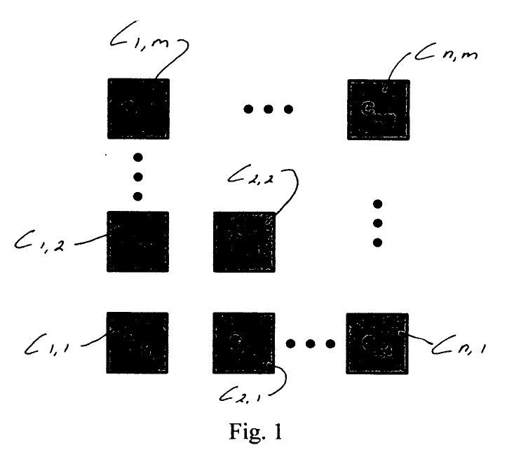

FIG. 1 shows a sensor array. A plurality of sensors, C_{i,m} through C_{n,1} are coupled together. Each of the members of the plurality of sensors can be capacitive sensors, inductive sensors, ultrasonic sensors, optical sensors, radio frequency sensors and/or other types of sensors. Each of the members of the plurality of sensors can be to measures thickness, density, viscosity and/or other types of state variables. The plurality of sensors can define an m by n substantially planar array, where m and n are both integers greater than or equal to 2.

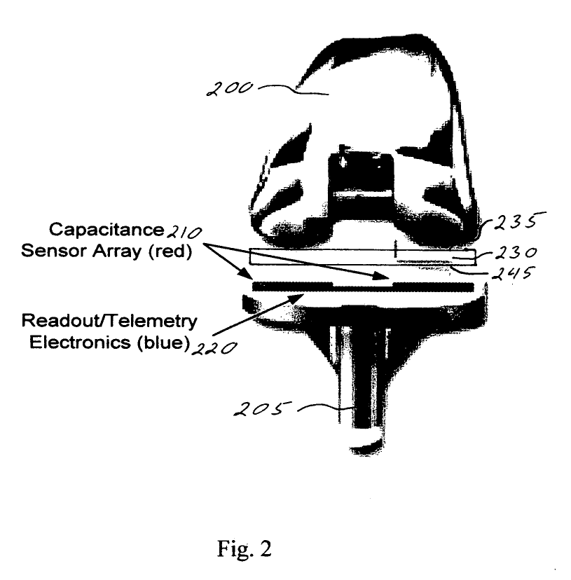

FIG. 2 illustrates placement of a sensor array in an artificial knee prosthesis. Of course, the invention can be deployed in the context of other orthopedic implants, in the context of other kinds of implants, or even in non-implant contexts.

Referring to FIG. 2, the artificial knee prosthesis includes a femoral portion 200. The artificial knee prosthesis includes a tibial portion 205 that is intended to be connected in vivo to the femoral portion 200. The tibial portion 205 includes a capacitance sensor array 210. The tibial portion 205 includes a readout/telemetry electronics layer that is electrically coupled to the capacitance sensor array 210. The femoral portion 200 can be coupled to a wear element 230 having an articulating surface 235. The wear element 240 can include another articulating surface 245. This configuration enables measuring a dimension in a direction that defines a path that passes through an articulating surface of a wear element of the orthopedic implant using at least one thickness sensor. In this configuration, the path also passes through another articulating surface of the wear element of the orthopedic implant.

Since the capacitance sensor array 210 is integrated with readout electronics 220, the invention allows for pixelated determination of wear at the friction surfaces (articulated surface). In a preferred configuration of the invention, the metal femoral portion of the prosthesis serves as a common node (or common capacitor plate) allowing accurate measurement of the distance between the polymer-embedded plates and the common node. In this configuration, the wear of the friction surfaces reduces the distance between the sensing plates as the capacitance of a parallel plate capacitor is linearly dependent on the distance between the plates. Neglecting fringing fields, this relationship is given by

$$C = \frac{ɛ A}{d} , \qquad ( 1 )$$

where ε is the effective dielectric constant of the material between the plates, A is the area of the plate, and d is the distance between the plates. Polymer thickness d is a direct indicator of wear.

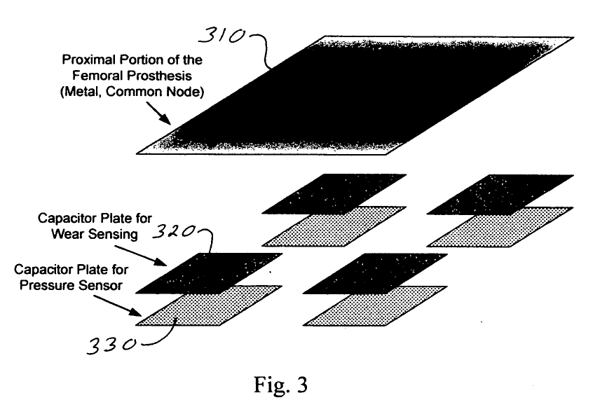

In addition, there is a so-called ‘fringe’ capacitance caused by the electric field emanating from the edges of the capacitor plate to the surrounding surfaces [13-14]. Similar to wear measurements, force measurements can be obtained by incorporating additional capacitive plates (or a common plate) in the tibial device. The additional capacitive plates can be spaced apart from the wear sensing capacitive plates with a (reversibly) compressible polymer. In this configuration, compression of the polymer will result in variations in the distance between the two plates that can be measured as a change in capacitance. An example of this third layer configuration is illustrated in FIG. 3. Also, it may be advantageous to incorporate in any of the capacitive measurements one or more fixed capacitive structures (structures whose dielectric spacing or dielectric values will not change with wear or pressure) for calibration, system testing, or system monitoring.

Referring to FIG. 3, with regard to the artificial knee context, a proximal portion 310 of the femoral prosthesis can be made of metal (e.g., titanium alloy) and function as a common node. A first set of thickness sensors includes a plurality of capacitor plates 320 for wear sensing. The plurality of capacitor plates 320 is separated from the proximal portion by one or more wear elements having one or more articulating (frictional) surfaces. In this embodiment, a second set of pressure sensors includes a plurality of capacitor plates 330 for pressure sensing. The plurality of capacitor plates 330 is separated from the plurality of capacitor plates 320 by one or more dielectrics. In preferred embodiments, an elastic material (e.g., polymer) is utilized to dielectrically separate the plates and reversibly measure force(s)/pressure(s)/stress(es) as a function of compression/strain. The circuits defined by the components 310, 320, 330 can be individually addressable. It is important to appreciate that the sensors can alternatively be based on coils (inductance), ultrasonic (time of flight) or other sensors, provided that the capability of measuring wear and/or pressure is provided.

Electronics Readout Configuration

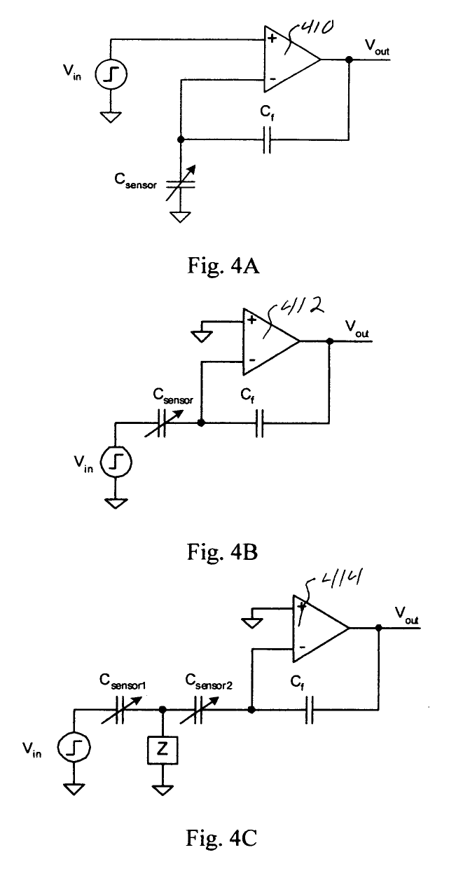

There are a number of different readout techniques that have been shown effective for use with capacitance-based sensors. Using the configuration shown in FIGS. 2 and 3, the wear sensing capacitors have one common plate (or node) formed by the tibial device. Several candidate sensor interfacing architectures are shown schematically in FIGS. 4A-4C.

Referring to FIG. 4A, an operational amplifier 410 is electrically coupled by a positive input to a voltage source Vᵢₙ. The operational amplifier is also electrically coupled to a capacitive sensor Cₛₑₙₛₒᵣ that provides an input that is a function of wear and/or pressure. A feedback capacitor C_{f} is electrically coupled between the capacitive sensor and an output of the operational amplifier 410. If a plurality of sensors are individually addressable, then a given sensor interfacing architecture can be switched between sensors.

Still referring to FIG. 4A, the capacitance sensor sets the low frequency gain of the circuit given by

$$\frac{Vout}{Vin} = 1 + \frac{{C}_{sensor}}{{C}_{f}} . \qquad ( 2 )$$

where Vin is a voltage applied to an operational amplifier (e.g., step function), Vout is a voltage from the operational amplifier, Cₛₑₙₛₒᵣ is a measured capacitance of a pressure sensor (variable; e.g., as a function of thickness, pressure, etc.) and C_{f} is a reference (e.g., feedback) capacitance. By adding switch elements, each capacitor sensor in a set of sensors can be individually addressed. Switching of one and/or a plurality of sensors in the overall network that includes the set of sensors can enable the measurement of individual and nearest neighbor parasitic capacitances and associated crosstalk between sensing elements. For instance, the parasitic capacitance with regard to each of the adjacent sensors nearest neighbors can be characterized and used to adjust (normalize) measured capacitance of one or more sensors. The implementation of the invention via an architecture composed of a multi-channel set of sensor amplifiers for wear and force sensing, feedback capacitors, switch elements, and control and support electronics is completely compatible with common low-power, low-voltage, integrated circuit fabrication processes.

In addition to the non-inverting voltage amplifier of FIG. 4A, an inverting configuration shown in FIG. 4B can be employed. Referring to FIG. 4B, an operational amplifier 412 is electrically coupled by a negative input to a voltage source Vᵢₙ via a capacitive sensor Cₛₑₙₛₒᵣ that provides an input that is a function of wear and/or pressure. A feedback capacitor C_{f} is electrically coupled between the capacitive sensor and an output of the operational amplifier 412. If a plurality of such sensors are individually addressable, then a given sensor interfacing architecture can be switched between sensors. In this embodiment, the transfer function is

$$\frac{Vout}{Vin} = - \frac{{C}_{sensor}}{{C}_{f}}$$

Referring to FIG. 4C, an operational amplifier 414 is electrically coupled by a negative input to a voltage source Vᵢₙ via a first capacitive sensor Cₛₑₙₛₒᵣ₁ and a second capacitive sensor Cₛₑₙₛₒᵣ₂. A feedback capacitor C_{f} is electrically coupled between the capacitive sensor and an output of the operational amplifier 414. Again, if a plurality of such sensors are individually addressable, then a given sensor interfacing architecture can be switched between sensors.

FIGS. 4A and 4B pertain to the common node drive configuration. FIG. 4C shows a configuration where a capacitive plate is driven with the opposite side of the implant being the common node (floating). This configuration has an impedance Z that may affect the measurement. Techniques based on optimization of the drive signal frequency content and signal processing can be employed to minimize this effect.

Embodiments of the invention can include incorporation of signal processing either in the implant or external to the patient (or a combination of these). For instance, a computer program can be used to compensate for parasitic impedance between sensors and thereby provide improved response of individual sensors by minimizing the effects of adjacent sensors. An embodiment of the invention can also utilize data processing methods that transform signals from raw data to (pre)processed data. For example, sensor outputs can be accumulated (e.g., integrated) and/or statistically processed (e.g., averaged, smoothed, etc.). Embodiments of the invention can be combined with instrumentation to obtain state variable information to actuate interconnected discrete hardware elements. For instance, an embodiment of the invention can include the use of temperature and/or vibration sensors to control the rate of data acquisition/transmission and drive characteristics where sensors requiring drive signals are employed.

Assessment of the soft tissue and bone surrounding the implant can be performed using a combination of optical, ultrasonics-based, and vibration measurements. An embodiment of the invention can include monitoring with any combination of optical, ultrasonic, and/or vibration sensors that are located peripherally with regard to a sensor set to probe into the soft tissue and/or bone surrounding the implant.

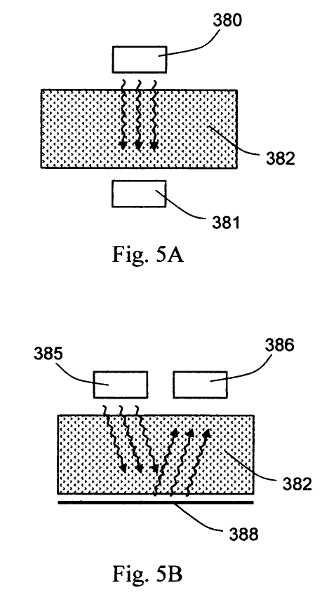

Thickness measurement of wear elements can be performed using optical absorption. For example, a nondispersive infrared light source can be chosen such that a portion of the light is absorbed by a polymeric wear element. The amplitude of the transmitted light can then be related to the thickness of the wear element. Referring to FIG. 5A, the light from source 380 is absorbed by the wear material 382. The light intensity at the detector 381 can be related to the thickness of the wear material. Alternatively, a similar measurement can be made using reflectance techniques as shown in FIG. 5B. Here the light emitter 385 and detector 386 are located on one side of the wear material 382. The light is reflected from the reflective surface 388.

Optical techniques can also be employed in the context of the invention to monitor the condition or formation of soft tissue inflammation. Inflamed tissue is characterized by fluid accumulation primarily in the interstitial spaces. This results in an increase in local tissue (soft-tissue) volume at the infection site detectable by a decrease in the measured tissue optical density or by the increase in the water absorption due to edema.

Optical techniques can be employed in the context of the invention to monitor the condition or formation of infection. Infection causative agents can be detected by optical means using DNA/antibody coated probes that bind to specific pathogens or toxins generating detectable optical signals or detected non specifically by the changes in scattering due to their presence. In addition, specific molecular species associated with infection and inflammatory processes can be measured using optical techniques.

Optical techniques can be employed in the context of the invention to monitor the condition or formation of scar tissue. Scar tissue is characterized by the presence of collagen-which has a very distinct auto-fluorescence signature that is detectable with a multi-spectral optical sensor[5,6]

Ultrasonic techniques can be utilized to detect the implant associated conditions such as bone mass deposition. Bone mass changes are detectable by attenuation changes in an ultrasound signal.

Ultrasonic techniques can be utilized to detect the implant associated conditions such as bone cement condition (deterioration). Bone cement changes (deterioration) will also be detectable by attenuation changes in an ultrasound signal[7].

Ultrasonic techniques can be utilized to detect the implant associated conditions such as long-term wear (thinning) of the implant by utilizing time-domain reflectometry.

Ultrasonic techniques can be utilized to detect the implant associated conditions such as real-time compression of the implant wear material by utilizing time-domain reflectometry.

The optical and/or ultrasonic sensors can be placed on the perimeter of the prosthesis where optical and ultrasonic access to the surrounding tissue can be established. Also, the optical and/or ultrasonic sensors can be located elsewhere on the prosthesis, for example to monitor the condition and/or performance of the prosthesis, the wear sensor(s) and/or the force/pressure sensor(s).

Instrument System

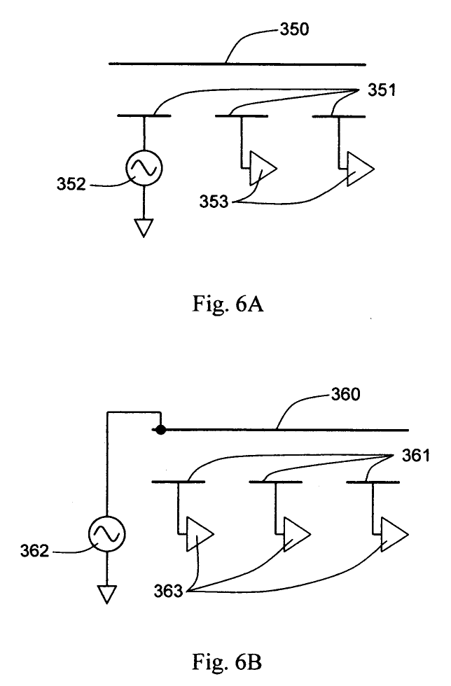

The individual sensors can be arranged into sets that define one or more arrays. When arranged in an array, the sensors can be termed pixilated sensors. The pixelated sensors can be configured in a number of ways to allow measurement of wear in the implant device. In FIG. 6A, one capacitor plate is driven by source 352. The wear material forms a dielectric between sensor plates 351 and a common capacitance plate or node 350. Capacitive signals are detected by amplifiers 353. In FIG. 6B, the common capacitive plate 360 is driven by oscillator 362 minimizing the effect of impedance loading of common node 350. Capacitive signals are detected on 361 and amplified by 363.

The sensing methods described above can be integrated with control and data telemetry electronics to provide a highly miniaturized low-power sensing system. Outputs from the multiple force and wear sensors can be digitized, and a data packet including sensor data, unit identification, etcetera can be transmitted to a localized receiver. Many options exist for the data telemetry including straight-forward amplitude modulation or frequency modulation, or more robust techniques employing spread spectrum. The power requirements of the sensor system depend on a number of factors including channel number, data acquisition rate, level of integrated signal processing, and data telemetry format. Options for implant powering include the use of an internal battery, inductive power coupling, or a combination of the two.

The invention can enable high-resolution pixelated sensing of wear and pressure, such as from approximately 1 micron to approximately 1 cm, preferably from approximately 10 microns to approximately 1 mm. The pixels can be defined by the spatial configuration of one or more associated sensor unit cell(s).

The invention can enable direct measurement of wear rather than a direct indication of force that can be used to solve for wear. The measurement of capacitance across wear element(s), inversely proportional to remaining wear element (and therefore wear) is an important aspect of the invention.

The invention can utilize the prosthetic elements as part of the sensing ‘circuit’ (e.g. for the knee prosthetic device, either the femoral implant, or the tibial implant (both metal), or both. The invention can also utilize the prosthetic elements for housing data storage, data processing, signal processing and/or signal transmission/reception elements.

The invention can enable dual use of the sensor plates. Sensor plates may be used both for sensing and for communications. The sensor plates may be configured to operate as either part of the sensing array or as communication devices. The two functions may be performed sequentially by switching control between sensing and communicating with regard to time separation. These two functions may be performed in parallel and separated in frequency allowing simultaneous functioning of both sensing and communication.

The sensor plates may be configured for use as a planar patch antenna with the tibial plate (metal portion), the femoral component (metal) or both acting as an image plane. The tibial plate (metal portion) or femoral component (metal portion) may be used as a transmitting antenna for communication of data to a receiving antenna located outside of the body. Similarly, the invention can include the use of a transmitter located outside the body to address the sensor, storage, processing and/or communication components of the implant.

The device may utilize the planar sensing plates for unmodulated baseband capacitive communications. Furthermore, modulation may be applied to these waveforms including BPSK, OOK, QPSK, ultrawideband, and other standard modulation/transmission formats.

The invention can include the use of inductive sensors. The inductive sensors (if utilized) may be employed for inductive communications with an externally place antenna or coil.

The invention may incorporate additional capacitive plates or inductive coils (in addition to those used for sensing) to enable data telemetry function. The telemetry function can be one-way or two-way.

The invention enables measurement of parameters indicating the presence or absence of infection. This may be implemented as a temperature sensor that measures small temperature variations that may be indicative of a localized infection or other immunological activity.

The invention enables measurement of surrounding tissue condition and infection status using single or multiple wavelength optical absorption spectroscopy. Polarization techniques may provide optimized discrimination. The invention enables measurement of surrounding bone condition and bone cement condition using miniature ultrasonic transducers.

The invention enables the incorporation of piezo-based sensors for force measurement. These piezo-based sensors may be used in the place of the pixelated capacitive sensors or can be stacked with the capacitance-based sensors.

The invention enables the use of ultrasonic-based sensors in addition to or in place of the capacitance-based sensors for direct wear measurement. An acoustic signal emitted from the sensor can pass through a polymer plate, reflect off of the femoral metal component (in the specific case of the knee prosthesis) and be detected by the sensor array. Time domain reflectometry (TDR) can then be employed to measure the thickness of the polymer and directly determine wear status. The phase of the reflected acoustic waveform may also be used to determine polymer spacer thickness. The use of acoustic TDR also allows for acoustic telemetry.

The invention enables the use of MEMs-based pressure sensors in addition to or in place of the capacitance-based sensors for force detection. These may be in the form of coated cantilevers or membrane-based sensors.

The invention enables the use of inductive sensors where inductive coils are used in place of or in addition to the capacitance-based plates. The inductance of these inductive coils will vary as the femoral portion of the implant (metal) is moved closer or farther from the coils. This will enable a direct determination of polymer spacer thickness (directly indicating wear) using inductance-based measurement techniques including RLC oscillators, L division, and LC shaping networks using zero-crossing techniques. In RLC oscillators, the R and C are fixed elements and L is the sensor inductance. Changes in L are indicated by changes in frequency allowing the polymer thickness to be approximated. L division employs two inductors placed in series. One L is a fixed reference device with the other is the sensor. The string is driven by a shaped pulse on one side and the pulse is measured between the two devices where changes in the sensor L are indicated by changes in the pulse characteristics.

The invention enables the use of capacitance sensor dielectric enhancement. In the capacitance-based case, stress-related mechanical compression will cause changes in both the dielectric properties of the media between the plates (joint polymer material) and plate separation. In addition, the acoustic velocity of propagation of the polymer will change as a function of pressure. These components can be utilized to provide increased sensitivity for both force and wear (thickness of polymer) monitoring. Wear will be observed as a long-term shift in the signal baseline while stress will involve temporal variations in capacitance.

The invention enables improved temperature tolerance. Temperature effects associated with capacitance-based monitoring will be minimized as temperature variations are limited by the damping thermal mass of the human body.

The invention can incorporate sensor node switching for pixilated sensor control allowing multiplexing of sensors to sensor interfacing/readout electronics. The invention can incorporate sensor node switching and signal processing allowing minimization of the effects of adjacent sensors on each pixel measurement.

The invention can be integrated with measurement, signal processing, and data telemetry electronics. Options for implant powering include the use of an internal battery, inductive power coupling, or a combination of the two.

The invention can incorporate different sensor types for wear and pressure sensing including capacitance-based, inductive based, piezo-based, and MEMs-based pressure sensors. The invention may incorporate ultrasonic-based sensors implementing time domain reflectometry (TDR) techniques for thickness measurement. The invention can incorporate of additional sensors for immunological assessment (infection, rejection, tissue encapsulation, scarring) including temperature sensors, MEMs-based sensors, and optical sensors. The invention can incorporate additional sensors for assessing integration of the implant with surrounding soft tissue and bone including optical and ultrasonic sensors.

Options for data telemetry including inductive, capacitive, optical, acoustic, and RF-modulated approaches including spread spectrum (direct sequence or frequency hopping approaches) and hybrid spread spectrum approaches. The telemetry can include real-time continuous reporting, periodic reporting, or reporting only when requested (e.g., polled). The invention may be configured with an internal receiver allowing programmability using one of the aforementioned communications methods.

An embodiment of the invention can also be included in a kit-of-parts. The kit-of-parts can include some, or all, of the components that an embodiment of the invention includes. The kit-of-parts can be an in-the-field retrofit kit-of-parts to improve existing systems that are capable of incorporating an embodiment of the invention. The kit-of-parts can include software, firmware and/or hardware for carrying out an embodiment of the invention. The kit-of-parts can also contain instructions for practicing an embodiment of the invention. Unless otherwise specified, the components, software, firmware, hardware and/or instructions of the kit-of-parts can be the same as those used in an embodiment of the invention.

### Practical Applications

A practical application of an embodiment of the invention that has value within the technological arts is for orthopedic implants. The invention is suitable for use with many different implant types including artificial knee (tibial plate, patella), hip, shoulder, and elbow joints, and may find use in spinal or other applications where bone is involved. There are virtually innumerable uses for an embodiment of the invention, all of which need not be detailed here.

### Advantages

Embodiments of the invention can be cost effective and advantageous for at least the following reasons. The invention is a technique enabling accurate measurement of direct wear and force parameters that can be incorporated into both research and clinical implants for continuous or periodic wear and load assessment. In addition, the invention can incorporate different sensor types allowing monitoring of surrounding physiological parameters including tissue encapsulation, bone condition, and the presence of infection. Embodiments of the invention improve quality and/or reduce costs compared to previous approaches.

### Definitions

The term reactance is intended to mean opposition to alternating current by storage in an electrical field (by a capacitor) or in a magnetic field (by an inductor), measured in ohms. The term susceptance is intended to mean the reciprocal of reactance, measured in siemens. The term program and/or the phrase computer program are intended to mean a sequence of instructions designed for execution on a computer system (e.g., a program and/or computer program, may include a subroutine, a function, a procedure, an object method, an object implementation, an executable application, an applet, a servlet, a source code, an object code, a shared library/dynamic load library and/or other sequence of instructions designed for execution on a computer or computer system). The phrase radio frequency (RF) is intended to mean frequencies less than or equal to approximately 300 GHz as well as the infrared spectrum.

The term substantially is intended to mean largely but not necessarily wholly that which is specified. The term approximately is intended to mean at least close to a given value (e.g., within 10% of). The term generally is intended to mean at least approaching a given state. The term coupled is intended to mean connected, although not necessarily directly, and not necessarily mechanically. The term proximate, as used herein, is intended to mean close, near adjacent and/or coincident; and includes spatial situations where specified functions and/or results (if any) can be carried out and/or achieved. The term deploying is intended to mean designing, building, shipping, installing and/or operating.

The terms first or one, and the phrases at least a first or at least one, are intended to mean the singular or the plural unless it is clear from the intrinsic text of this document that it is meant otherwise. The terms second or another, and the phrases at least a second or at least another, are intended to mean the singular or the plural unless it is clear from the intrinsic text of this document that it is meant otherwise. Unless expressly stated to the contrary in the intrinsic text of this document, the term or is intended to mean an inclusive or and not an exclusive or. Specifically, a condition A or B is satisfied by any one of the following: A is true (or present) and B is false (or not present), A is false (or not present) and B is true (or present), and both A and B are true (or present). The terms a or an are employed for grammatical style and merely for convenience.

The term plurality is intended to mean two or more than two. The term any is intended to mean all applicable members of a set or at least a subset of all applicable members of the set. The term means, when followed by the term “for” is intended to mean hardware, firmware and/or software for achieving a result. The term step, when followed by the term “for” is intended to mean a (sub)method, (sub)process and/or (sub)routine for achieving the recited result.

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. The terms “consisting” (consists, consisted) and/or “composing” (composes, composed) are intended to mean closed language that does not leave the recited method, apparatus or composition to the inclusion of procedures, structure(s) and/or ingredient(s) other than those recited except for ancillaries, adjuncts and/or impurities ordinarily associated therewith. The recital of the term “essentially” along with the term “consisting” (consists, consisted) and/or “composing” (composes, composed), is intended to mean modified close language that leaves the recited method, apparatus and/or composition open only for the inclusion of unspecified procedure(s), structure(s) and/or ingredient(s) which do not materially affect the basic novel characteristics of the recited method, apparatus and/or composition.

Unless otherwise defined, all technical and scientific terms used herein have the same meaning as commonly understood by one of ordinary skill in the art to which this invention belongs. In case of conflict, the present specification, including definitions, will control.

### Conclusion

The described embodiments and examples are illustrative only and not intended to be limiting.

Although embodiments of the invention can be implemented separately, embodiments of the invention may be integrated into the system(s) with which they are associated. All the embodiments of the invention disclosed herein can be made and used without undue experimentation in light of the disclosure. Although the best mode of the invention contemplated by the inventor(s) is disclosed, embodiments of the invention are not limited thereto. Embodiments of the invention are not limited by theoretical statements (if any) recited herein. The individual steps of embodiments of the invention need not be performed in the disclosed manner, or combined in the disclosed sequences, but may be performed in any and all manner and/or combined in any and all sequences. The individual components of embodiments of the invention need not be formed in the disclosed shapes, or combined in the disclosed configurations, but could be provided in any and all shapes, and/or combined in any and all configurations.

It can be appreciated by those of ordinary skill in the art to which embodiments of the invention pertain that various substitutions, modifications, additions and/or rearrangements of the features of embodiments of the invention may be made without deviating from the spirit and/or scope of the underlying inventive concept. All the disclosed elements and features of each disclosed embodiment can be combined with, or substituted for, the disclosed elements and features of every other disclosed embodiment except where such elements or features are mutually exclusive. The spirit and/or scope of the underlying inventive concept as defined by the appended claims and their equivalents cover all such substitutions, modifications, additions and/or rearrangements.

The appended claims are not to be interpreted as including means-plus-function limitations, unless such a limitation is explicitly recited in a given claim using the phrase(s) “means for” and/or “step for.” Subgeneric embodiments of the invention are delineated by the appended independent claims and their equivalents. Specific embodiments of the invention are differentiated by the appended dependent claims and their equivalents.

### REFERENCES

1. Kenton R. Kaufman, PhD, Nebosja Kovacevic, MSAE, Steven E, Irby, B S, and Clifford W. Colwell, M D, Instrumented Implant for Measuring Tibiofemoral Forces, Journal of Biomechanics, Vol 29, No. 5, pp 667-671 (1996).
2. U.S. Pat. No. 5,197,488 (March, 1993), Nebojsa Kovacevic—Knee Joint Load Measuring Instrument and Joint Prosthesis.
3. G. D. Ehrlich, et al., “Development of intelligent implants for biofilm prevention in orthopedic prostheses,” Bio-, Micro-, and Nanosystems, 2003. ASM Conferences, Jul. 7-10, 2003, Page 14.
4. J. L. Kadurugamuwa, L. Sin, E. Albert, J. Yu, K. Francis, M. DeBoer, M. Rubin, C. Bellinger-Kawahara, T. R. Parr, Jr., and P. R. Contag, “Direct Continuous Method for Monitoring Biofilm infection in a Mouse Model,” Infection and Immunity 71(2) :882-890 (2003).
5. R. Richards-Kortum, and E. Sevick-Muraca, “Quantitative optical spectroscopy for tissue diagnosis,” Annu Rev Phys Chem. 47:555-606 (1996).
6. J. F. White, J. A. Werkmeister, I. A. Darby, T. Bisucci, D. E. Birk, and J. A. Ramshaw., “Collagen fibril formation in a wound healing model” J Struct Biol. 137(1-2):23-30 (2002).
7. A. M. Viano, J. A. Auwarter, J. Y. Rho, and B. K. Hoffmeister, “Ultrasonic characterization of the curing process of hydroxyapatite-modified bone cement,” J Biomed Mater Res. 56(4): 593-599 (2001).
8. Burny, F.; Donkerwolcke, M.; Moulart, F.; Bourgois, R.; Puers, R.; Van Schuylenbergh, K.; Barbosa, M.; Paiva, O.; Rodes, F.; Begueret, J. B.; Lawes, P. “Concept, design and fabrication of smart orthopedic implants” Medical Engineering & Physics, v 22, n 7, September 2000, pp. 469-79.
9. R. Puers, M. Catrysse, G. Vandevoorde, R. J. Collier, E. Louridas, F. Burny, M. Donkerwolcke, F. Moulart, “A telemetry system for the detection of hip prosthesis loosening by vibration analysis,” Sensors and Actuators, 85, 2000 pp. 42-47.
10. Amirouche, L. Aram, M. Gonzalez, R Giachetti, C. Mahr, “The Fitting of the Human Joint Through Micro Implanted Sensors,” Annual International EEE-EMBS Special Topic Conference on Microtechnologies in Medicine & Biology, Poster 137, Oct. 12-14, 2000, Lyon, France.
11. Taylor S J G, “A Telemetry System For Measurement Of Forces In Massive Orthopedic Implants in vivo,” 18th Annual International Conference of the IEEE Engineering in Medicine and Biology Society, Amsterdam 1996, 1.7.2: Telemetry I1, pp. 290-292.
12. F. E. Sauser, D. Glos, D. Bylski-Austrow, and I. Papautsky, “Pressure Sensors for In Vivo Measurements on Spinal Growth Plates,” Proceedings of the 26th Annual International Conference of the IEEE EMBS San Francisco, Calif., USA, Sep. 1-5, 2004, pp. 2430-2433.
13. T. Sakurai and K. Tamaru, “Simple Formulas for Two- and Three-Dimensional Capacitances,” IEEE Trans. Electron Devices, v. ED-30, no. 2, February 1983, pp. 183-185.
14. C. P. Yuan and T. N. Trick, “A Simple Formula for the Estimation of the Capacitance of Two-Dimensional Interconnects in VLSI Circuits,” IEEE Electron Device Lett., v. EDL-3, 1982, pp. 391-393.

What is claimed is:

1. A method, comprising characterizing wear of an orthopedic implant including measuring a dimension in a direction that defines a path that passes through an articulating surface of a wear element of the orthopedic implant using at least one thickness sensor.

2. The method of claim 1, wherein the path passes through another articulating surface of the orthopedic implant.

3. The method of claim 1, wherein using at least one thickness sensor includes using at least one capacitive sensor.

4. The method of claim 3, further comprising performing with a fixed capacitor at least one purpose selected from the group consisting of calibrating, testing and monitoring.

5. The method of claim 1, wherein using at least one thickness sensor includes using at least one inductive sensor.

6. The method of claim 5, further comprising performing with a fixed inductor at least one member selected from the group consisting of calibrating, testing and monitoring.

7. The method of claim 1, wherein using at least one thickness sensor includes using at least one ultrasonic sensor.

8. The method of claim 1, wherein using at least one thickness sensor includes using at least one optical-based sensor.

9. The method of claim 1, wherein using at least one thickness sensor includes using a plurality of thickness sensors.

10. The method of claim 9, wherein the plurality of thickness sensors include a plurality of capacitors, and, further comprising communicating data to a receiving antenna, located outside a body in which the orthopedic implant is located, by configuring the plurality of capacitors as a planar patch antenna having at least one metal portion acting as an image plane selected from a group consisting of a tibial plate and a femoral component.

11. The method of claim 9, wherein using the plurality of thickness sensors includes using a plurality of thickness sensors that define an m by n substantially planar array, where m and n are both integers greater than or equal to 2.

12. The method of claim 9, wherein using the plurality of thickness sensors includes using a plurality of capacitive sensors that share a common plate.

13. The method of claim 9, further comprising characterizing forces within the orthopedic implant using a plurality of pressure sensors coupled to the plurality of thickness sensors.

14. The method of claim 13, wherein using the plurality of pressure sensors includes using a plurality of pressure sensors that share a common elastic layer.

15. The method of claim 13, wherein using the plurality of pressure sensors includes using a plurality of capacitive sensors.

16. The method of claim 15, wherein the plurality of capacitive sensors are individually addressable, and further comprising characterizing parasitic capacitance between at least two of the plurality of capacitive sensors.

17. The method of claim 13, wherein using the plurality of pressure sensors includes using a plurality of piezoelectric sensors.

18. The method of claim 13, wherein using the plurality of pressure sensors include using a plurality of inductive sensors.

19. The method of claim 18, wherein the plurality of inductive sensors are individually addressable, and further comprising characterizing parasitic inductance between at least two of the plurality of inductive sensors.

20. A method of periodically monitoring orthopedic implant wear comprising repeating the method of claim 1.

21. An apparatus, comprising an orthopedic implant including
a wear element having an articulating surface; and
at least one thickness sensor coupled to the wear element, the at least one thickness sensor measuring a dimension in a direction that defines a path that passes through the articulating surface of the wear element.

22. The apparatus of claim 21, wherein the orthopedic implant includes another articulating surface, the path passing through the another articulating surface.

23. The apparatus of claim 21, wherein the at least one thickness sensor includes at least one capacitive sensor.

24. The apparatus of claim 23, further comprising a fixed capacitor adapted to at least one purpose selected from the group consisting of calibrating, testing and monitoring.

25. The apparatus of claim 21, wherein the at least one thickness sensor includes at least one inductive sensor.

26. The apparatus of claim 25, further comprising a fixed inductor adapted to perform at least one function selected from the group consisting of calibrating, testing and monitoring.

27. The apparatus of claim 21, wherein the at least one thickness sensor includes at least one ultrasonic sensor.

28. The apparatus of claim 21, wherein the at least one thickness sensor includes at least one optical sensor.

29. The apparatus of claim 21, wherein the at least one thickness sensor includes a plurality of thickness sensors.

30. The apparatus of claim 29, wherein the plurality of thickness sensors include a plurality of capacitors configured for use as a planar patch antenna having at least one metal portion acting as an image plane selected from a group consisting of a tibial plate and a femoral component.

31. The apparatus of claim 29, wherein the plurality of thickness sensors define an m by n substantially planar array, where m and n are both integers greater than or equal to 2.

32. The apparatus of claim 29, wherein the plurality of thickness sensors include a plurality of capacitive sensors that share a common plate.

33. The apparatus of claim 29, further comprising a plurality of pressure sensors coupled to the plurality of thickness sensors.

34. The apparatus of claim 33, wherein the plurality of pressure sensors share a common elastic layer.

35. The apparatus of claim 33, wherein the plurality of pressure sensors include a plurality of capacitive sensors.

36. The apparatus of claim 35, wherein the plurality of capacitive sensors are individually addressable to characterize parasitic capacitance between at least two of the plurality of capacitive sensors.

37. The apparatus of claim 33, wherein the plurality of pressure sensors include a plurality of piezoelectric sensors.

38. The apparatus of claim 33, wherein the plurality of pressure sensors include a plurality of inductive sensors.

39. The apparatus of claim 38, wherein the plurality of inductive sensors are individually addressable to characterize parasitic inductance between at least two of the plurality of inductive sensors.

40. The apparatus of claim 21, further comprising at least one optical sensor coupled to the at least one thickness sensor, wherein the at least one optical sensor characterizes tissue adjacent the orthopedic implant.

41. The apparatus of claim 21, further comprising at least one ultrasonic sensor coupled to the at least one thickness sensor, wherein the at least one ultrasonic sensor characterizes tissue adjacent the orthopedic implant.

42. A method, comprising characterizing forces within an orthopedic implant including using a plurality of individually addressable pressure sensors including measuring parasitic impedance between at least two of the plurality of individually addressable pressure sensors.

43. The method of claim 42, wherein using the plurality of individually addressable pressure sensors includes using at least two capacitive sensors and characterizing parasitic impedance includes characterizing parasitic capacitance between the at least two capacitive sensors.

44. The method of claim 42, wherein using the plurality of individually addressable pressure sensors includes using at least two inductive sensors and characterizing parasitic impedance includes characterizing parasitic inductance between the at least two capacitive sensors.

45. The method of claim 42, wherein using the plurality of individually addressable pressure sensors includes using a plurality of individually addressable pressure sensors that define an m by n substantially planar array, where m and n are both integers greater than or equal to 2.

46. The method of claim 42, wherein using the plurality of individually addressable pressure sensors includes using a plurality of individually addressable pressure sensors that share a common elastic layer.

47. An apparatus for performing the method of claim 42.
